(19) United States
(12) Patent Application Publication
(10) Pub. No.: US 2026/0007359 A1
(43) Pub. Date: Jan. 8, 2026

(54) THERMAL IMAGING SYSTEM FOR DETECTION OF EARLY STAGES OF DIABETIC FOOT ULCERS

(71) Applicants: Meshari Alwashmi, Riyadh (SA); Mustafa Alghali Elsaid Muhammed, Riyadh (SA)

(72) Inventors: Meshari Alwashmi, Riyadh (SA); Mustafa Alghali Elsaid Muhammed, Riyadh (SA)

(21) Appl. No.: 18/763,566

(22) Filed: Jul. 3, 2024

(51) Int. Cl. A61B 5/00; A61B 5/01; G06T 7/00; G16H 15/00; G16H 40/67; G16H 50/30

(52) U.S. Cl. CPC A61B 5/445; A61B 5/015; A61B 5/4538; A61B 5/7275; A61B 5/748; G06T 7/0014; G16H 15/00; G16H 40/67; G16H 50/30; G06T 2200/24; G06T 2207/10048; G06T 2207/20084; G06T 2207/30096

(57) ABSTRACT

A thermal imaging system for detection of diabetic foot ulcers including a mobile device having a graphical user interface. The thermal imaging system includes a thermal camera in communication with the mobile device for obtaining thermal images of patient feet and sending the thermal images to the mobile device. The thermal imaging system includes an application on the mobile device having instruction for analyzing the thermal images using a temperature condition index during analysis. The application provides data results on the interactive dashboard of the graphical user interface. The instruction for analyzing the thermal images include AI-driven analysis for delivering rapid results, reducing need for extensive medical expertise. Following analysis, the application generates a detailed report including information on categorization of the diabetic foot condition, risk level, recommended screening frequency and suggested actions.

### COPYRIGHT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

Trademarks used in the disclosure of the invention, and the applicants, make no claim to any trademarks referenced.

### BACKGROUND OF THE INVENTION

### 1) Field of the Invention

The invention relates to the field of thermal scans for use in diabetic foot care and, more particularly to thermal scans used in the detection of diabetic foot ulcers.

### 2) Description of Related Art

Diabetes Mellitus-Diabetes affects 1 in 10 adults worldwide, currently around 537 million adults. This number is predicted to rise to 643 million by 2030 and 783 million by 2045. In the Middle East and North Africa (MENA) region, the prevalence is higher as it affects 1 in 6 adults or about 73 million in that region. Despite advances in medical therapies, the prevalence of diabetes mellitus and diabetes-related complications continues to rise.

One of the most common complications of diabetes is diabetic foot ulcer (DFU). It is estimated that one-third of people with diabetes will develop a DFU during their lifetime. Emphasized the alarming statistic that every 20 seconds, a lower limb is amputated due to complications of diabetes, with 85% of these amputations preceded by a foot ulcer. The mortality risk at 5 years for individuals with diabetic foot ulcers is 2.5 times higher than for those without ulcers. DFUs and lower extremity amputations (LEAs) are not only markers of poor health but also independent risk factors associated with premature death. Unfortunately, even after a DFU has been resolved, recurrence is common and is estimated to be 40% within one year, 60% within three years, and 65% within five years. Lower limb amputation is the most severe and costly outcome if DFU complications persist.

Diabetic foot ulcers (DFUs) constitute a significant and multifaceted economic burden within healthcare systems worldwide. Diabetes foot care costs are the single largest category of diabetes-related medical costs. The cost of care for patients with a foot ulcer is 5.4 times higher than that for diabetic patients without ulcers, accentuating the heightened financial burden linked to DFUs.

Prevention of these lower limb complications could lead to a significant positive impact on health outcomes and significant cost savings. Unfortunately, current tools for detecting DFU have limited scalability in terms of time efficiency and practicality. These findings collectively emphasize the urgent need for comprehensive prevention, specialized care teams, and limb salvage strategies to mitigate the economic and health-related consequences of DFUs.

There are current challenges in early detection of diabetic foot ulcers. Unfortunately, DFUs can be difficult to detect, especially in the early stages. This is due to a number of factors, including inconsistent screening guidelines, limited awareness among patients and providers, and the frequent occurrence of silent or non-standard symptoms. Inconsistent screening guidelines can lead to confusion and variation in practice among healthcare providers. Many people with diabetes are not aware of the risk factors for DFUs or the signs and symptoms to look for. Healthcare providers may also not be aware of the latest screening guidelines or best practices for DFU prevention and management. In the diagnosis of DFUs, clinicians have long depended on traditional methods, each bearing distinct advantages and limitations.

In examining a first of these diagnostic tools, the monofilament test is not always reliable for early detection of diabetic foot ulcers because it primarily identifies loss of protective sensation, missing other risk factors like arterial insufficiency and structural deformities. The test's accuracy can also be influenced by the technique of the administrator, patient factors like swelling and temperature, and may not pick up early or intermittent neuropathy symptoms. For a more comprehensive assessment, clinicians often use the monofilament test in conjunction with other evaluations and tests.

Foot ulcers can be exacerbated by underlying peripheral arterial disease (PAD). The ankle-brachial index (ABI) is a widely used, non-invasive method for diagnosing PAD. However, its accuracy is debated, especially in patients with exertional leg pain. Furthermore, it is not useful to detect PAD among patients with diabetes because calcified vessels can distort results. This raises concerns about the potential for an unreliable ABI to overlook those at heightened risk for foot ulcers, emphasizing the importance of a comprehensive assessment in diagnosing PAD and determining ulcer risk.

Duplex ultrasonography (DUS) is an imaging test used to diagnose and monitor PAD. DUS can accurately identify the location and severity of narrowing or blockage in arteries. However, some artery segments are difficult to visualize with DUS, especially in the lower limb, and the results may not be fully reliable. Additionally, DUS is not well-suited for people with diabetes because their calcified vessels are less flexible and more difficult to see with ultrasound. For this reason, other testing methods are often more reliable for diagnosing PAD in people with diabetes.

Angiography is the most accurate way to diagnose DFUs by using a special type of X-ray called an angiogram. Repeated exposure to X-rays can be harmful to patients' health. The test is expensive and time-consuming. Often, patients need to be hospitalized overnight for the procedure. To improve the early detection of DFUs, it is important to develop and implement universal screening guidelines, increase awareness among patients and providers, and develop and implement better screening tools and methods.

### BRIEF SUMMARY OF THE INVENTION

The instant invention in one form is directed to a thermal imaging system for detection of diabetic foot ulcers, the system including a mobile device including a graphical user interface, a thermal camera in communication with the mobile device, the thermal camera for obtaining thermal images of patient feet and sending the thermal images to the mobile device and an application on the mobile device. The application includes instruction for analyzing the thermal images using a temperature condition index during analysis. The application provides data results on the graphical user interface. The data results are provided on an interactive dashboard. The interactive dashboard may include a current diagnosis and may include medical risk results including risk of amputation. The thermal imaging device may be securable to the mobile device. The instruction for analyzing the thermal images may include AI-driven analysis for delivering rapid results, reducing need for extensive medical expertise and easing workload of healthcare professionals. Following analysis, the application may generate a detailed report including information on categorization of the diabetic foot condition, risk level, recommended screening frequency, and suggested actions and the thermal imaging system may be integratable with a healthcare system and wherein the report enables medical professionals to seamlessly incorporate results into a patient care plan. The mobile application may have an interactive dashboard including an economic impact dashboard, social impact dashboard, and an economic impact dashboard. A machine learning model may be accessible by the mobile device wherein machine learning algorithms analyze variations in temperature and image patterns which indicate potential health issues and wherein the machine learning models process large sets of data with high accuracy. The system may calculate risk factors and provides results of the risk factors on the graphical user interface. A scan report may be provided for a patient review or for further medical evaluation. The system may use a Paco's Curve during the analysis.

Another aspect of the system is directed to a method of using the thermal imaging system described in the paragraph above. The method includes initiating the mobile device application, obtaining patient information, obtaining scanned thermal images by scanning feet of the patient using the thermal camera and initializing analysis of the scanned thermal images using a temperature conditioning index. The application providing patient results to the graphical user interface of the mobile device.

Another aspect of the method includes initiating the mobile device application, obtaining patient information, obtaining scanned thermal images by scanning feet of the patient using the thermal camera and initializing analysis of the scanned thermal images using a temperature conditioning index. The method includes the mobile device accessing a neural network wherein data obtained from the analysis of the scanned thermal images is sent to the neural network for comparison to learned data stored on the neural network and wherein the neural network provides feedback data to the mobile device for determining final analysis of patient results, the application providing the final patient results to the graphical user interface of the mobile device.

Another aspect of the thermal imaging system for diabetic foot care includes a computer medium for processing information, the computer medium including storage for patient information, patient history and scan results and a computer processor for analyzing the patient information, the patient history and the scan results and for determining recommendations and a course of action.

Another aspect of the system of analysis using a thermal foot scan for identifying diabetic foot complications includes using thermal cameras, compatible with a mobile device for capturing detailed thermal images of patient feet. The system uses machine learning models for analyzing thermal images captured by the thermal camera, wherein algorithms on the mobile device detect variations in temperature and thermal patterns that indicate potential health issues, including processing large sets of data wherein the system ensures accuracy by implementing learned data from the machine learning models.

Another aspect of the system is directed to a thermal imaging system for detection of diabetic foot ulcers. The system includes a mobile device including a graphical user interface and a thermal camera in communication with the mobile device. The thermal camera is for obtaining thermal images of patient feet and sending the thermal images to the mobile device. The thermal imaging system includes an application on the mobile device for processing thermology data obtained by the thermal camera, a plurality of datasets accessible by the mobile device and a reporting service for delivering data results. The mobile device accesses at least one of the plurality of datasets and sends information to the at least one of the plurality of datasets. The data results may be provided on an interactive dashboard. The interactive dashboard may include medical risk results including risk of amputation. The reporting service may deliver the data results to the patient, the medical team of the patient or to a healthcare system database.

These and other objects, features, and advantages of the present invention will become more readily apparent from the attached drawings and the detailed description of the preferred embodiments, which follow.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. When reference is made to a reference numeral without specification to an existing sub-label, it is intended to refer to all such multiple similar components.

FIG. 1 shows a grayscale rendition of a thermal image of the lower extremities of a healthy subject;

FIG. 2 shows a grayscale rendition of a thermal image of the lower extremities of a first diabetic subject;

FIG. 3 shows a grayscale rendition of a thermal image of the lower extremities of a second diabetic subject;

FIG. 4 shows a thermal imaging device used in the thermal imaging system;

FIG. 5 shows a smartphone which is used in the thermal imaging system;

FIG. 6 shows a graph highlighting the risks of early versus late-stage detection of diabetic foot ulcers;

FIG. 7 shows a tablet visualizing a report of findings from a scan using the thermal imaging system according to the present invention;

FIG. 8 shows a visual representation of an economic impact dashboard;

FIG. 9 shows a visual representation of a social impact dashboard;

FIG. 10 shows data from a patient scan report;

FIG. 11 shows a non-thermal picture of a patient's feet;

FIG. 12 shows a first representation of feet in a temperature conditioning index;

FIG. 13 shows a second representation of feet in a temperature conditioning index;

FIG. 14 shows a third representation of feet in a temperature conditioning index;

FIG. 15 shows a fourth representation of feet in a temperature conditioning index;

FIG. 16 is graph showing a graph including Paco's Curve representation of a patient's right and left foot;

FIG. 17 is a diagram showing a preferred embodiment of the present thermal imaging system for detecting diabetic foot ulcers; and

FIG. 18 is a flowchart showing the steps of a method for using a thermal imaging system for the detection of diabetic foot ulcers.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION

While various aspects and features of certain embodiments have been summarized above, the following detailed description illustrates a few exemplary embodiments in further detail to enable one skilled in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art however that other embodiments of the present invention may be practiced without some of these specific details. Several embodiments are described herein, and while various features are ascribed to different embodiments, it should be appreciated that the features described with respect to one embodiment may be incorporated with other embodiments as well. By the same token however, no single feature or features of any described embodiment should be considered essential to every embodiment of the invention, as other embodiments of the invention may omit such features.

In this application the use of the singular includes the plural unless specifically stated otherwise and use of the terms “and” and “or” is equivalent to “and/or,” also referred to as “non-exclusive or” unless otherwise indicated. Moreover, the use of the term “including,” as well as other forms, such as “includes” and “included,” should be considered non-exclusive. Also, terms such as “element” or “component” encompass both elements and components including one unit and elements and components that include more than one unit, unless specifically stated otherwise.

Lastly, the terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

As this invention is susceptible to embodiments of many different forms, it is intended that the present disclosure be considered as an example of the principles of the invention and not intended to limit the invention to the specific embodiments shown and described.

The terms TFScan and the thermal imaging system are used interchangeably to mean a system which uses thermology and computer vision to detect diabetic foot ulcers.

The terms thermal imaging camera and thermal camera are used interchangeably to mean a device which captures thermal information and provides data output based on the captured thermal information. The thermal information is represented on a computer processing device in the form of a colored image having different colors representing the captured thermal information.

Medical thermography results from decades of research and development in the performance of infrared imaging equipment, standardization of technique, and clinical protocols for thermal imaging medical thermography visualize diseases which are not readily detected or monitored by other methods. Medical thermography is a fast, passive, non-contact and non-invasive imaging method that has been used by numerous peer-reviewed studies. It is currently used globally to screen, detect, and monitor diseases. The American Academy of Thermology (AAT) established guidelines for the use of thermography in the evaluation of diabetic patients. These guidelines provide recommendations for the use of thermal imaging in the detection and monitoring of diabetic neuropathy, including protocols for image acquisition and interpretation.

Medical Thermography has many advantages that could encourage widespread adoption. Thermal imaging is relatively inexpensive, compact, portable, involves no ionizing radiation, and requires little electric power. Recent technological breakthroughs have transformed large and expensive stationary cameras into portable and inexpensive solutions while maintaining quality imaging.

A major limitation of the current state of medical thermography is that even the most skilled human thermographer can only observe, analyze, and successfully interpret a limited number of thermograms. Computers, however, can process an image efficiently and extract useful information without tiring. Leveraging Artificial Intelligence (AI) algorithms, specifically, computer vision, can objectively observe the findings and minimize inter-observer variability.

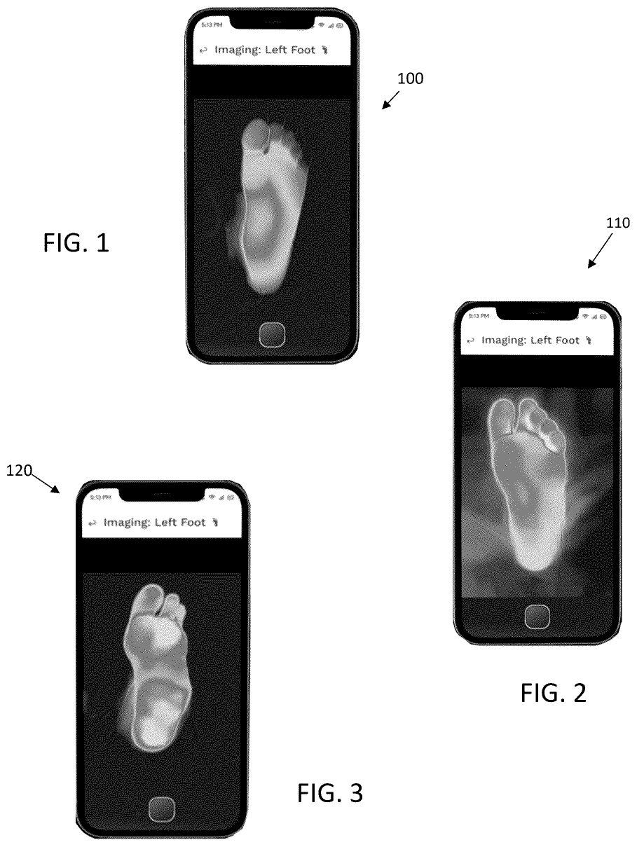

Ongoing progress in software image analysis and reduced reliance on human labor results in faster throughput and centralized processing. This can lead to increased thermographic accuracy and reliability. Nevertheless, computer-aided thermography will require high-level training and experience to ensure quality outcomes. Although thermology is the primary technology described, hyperspectral imaging may be used in place of or in combination with thermology. FIGS. 1-3 show grayscale renditions of thermal images on the graphical interface of the mobile device, the thermal images showing lower extremities of one healthy and two diabetic subjects, the thermal images of plantar region of the foot for healthy, at risk, and amputated subjects.

Thermography is helpful for the early detection of abnormalities of the foot by analyzing asymmetries and local temperature changes over time. Assessing temperature differences can enable the early detection of ulcers. Peripheral vascular disease (PVD) is a common complication of diabetes, which can result in alterations in blood flow that induce changes in skin temperature. These changes in skin temperature may also indicate tissue damage or inflammation resulting from trauma or excessive pressure. The etiology of these traumas is frequently related to moderate repetitive stress that goes unnoticed due to diabetic neuropathy (DPN). The application of thermal imaging for the detection of diabetic foot complications is based on the premise that variations in plantar temperature are associated with these types of complications. Furthermore, there appears to be a positive correlation between Body Mass Index and the risk of diabetic foot complications in patients with type 2 diabetes. Two systematic reviews concluded that the use of thermography to monitor plantar temperature is a promising tool in the prevention of foot ulcers.

The rapid development of handheld smartphone-based thermal infrared imagers presents a solution for detecting and monitoring DFUs. To address the lack of thermographers, practical computer vision algorithms are provided in the thermal imaging system to automate the process of image acquisition and analysis. The thermal cameras are preferably low-cost and easily available and aid in predicting risk of a patient developing foot ulcers. The thermal imaging system aids in saving patient limbs and patient lives.

AI and its applications are increasingly demonstrating promise in the detection and management of diabetic foot ulcers. Diabetes foot syndrome, with its lack of early symptoms and significant impact on patients' quality of life, benefits from the use of AI in timely screening and detection of risk for foot ulcers and possible amputations.

Studies show the potential of infrared thermography for detecting foot complications in diabetic patients. Several researchers demonstrated promising results in the detection of diabetic foot ulcers using machine learning techniques to analyze foot images. These findings suggest that AI's application to data derived from thermal plantar images yields promising results. AI has a significant potential to revolutionize the detection and management of diabetic foot ulcers. In this study, the thermal imaging system leverages AI technologies that are deployed on a smartphone-based thermal imager and application.

Early active intervention holds the key to significantly reducing foot ulcer incidence and amputations in people with diabetes. Therefore, early diagnosis and treatment of diabetic foot ulcers are crucial, as highlighted by the International Working Group on the Diabetic Foot. An annual foot exam is recommended to identify high-risk conditions, with more frequent assessments depending on individual findings. Patients with one or more high-risk foot conditions should receive professional diabetic foot care.

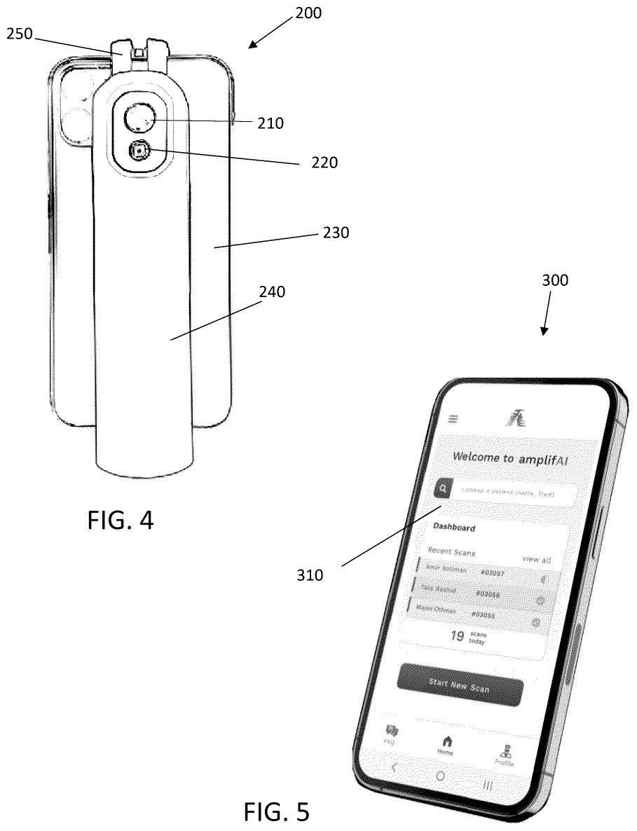

FIG. 4 shows one example of a thermal imaging system 200 with a mobile device 230 having a thermal camera 240 secured to the mobile device 230 with a clip 250. The thermal camera 240 includes an input sensor 220 and may include a light source 210 for capturing non-thermal images of a foot. The thermal imaging system includes a non-invasive system of thermal foot scans designed to identify diabetic foot complications at an early stage. The thermal imaging system preferably uses off-the-shelf thermal cameras so a user may use the mobile device application by securing the easily available thermal camera to the mobile device. The thermal camera may automatically submit images to be processed by the system algorithms. Although FIG. 4 shows an example of the thermal camera, the thermal camera does not require clips or attachment to a smartphone. Both FLIR and SEEK thermal cameras are examples of cameras that may be used in the system.

The mobile device shown in FIG. 5 is a smart phone 300 wherein the mobile device includes a graphical user interface 310 for displaying information and a computer medium for storing information and a processor for processing information for analysis of feet of a patient, the smart phone 300 in communication with a thermal imaging camera for capturing the thermal images of the feet of the patient. The thermal imaging system uses advanced machine learning models that analyze these thermal images wherein algorithms are specifically designed to detect variations in temperature and patterns that indicate potential health issues. By processing large sets of data with high accuracy, the thermal imaging system ensures its technology remains at the cutting edge of medical imaging and AI.

Diabetic foot ulcers should be diagnosed before becoming visible and reaching stage 3. The thermal imaging system takes this a step further by detecting these potentially devastating ulcers at an unprecedentedly early stage, as shown in FIG. 3. By identifying subtle risk indicators before clinical symptoms appear, the thermal imaging system opens a window for proactive intervention and preventative care, making a truly transformative impact on diabetic foot health.

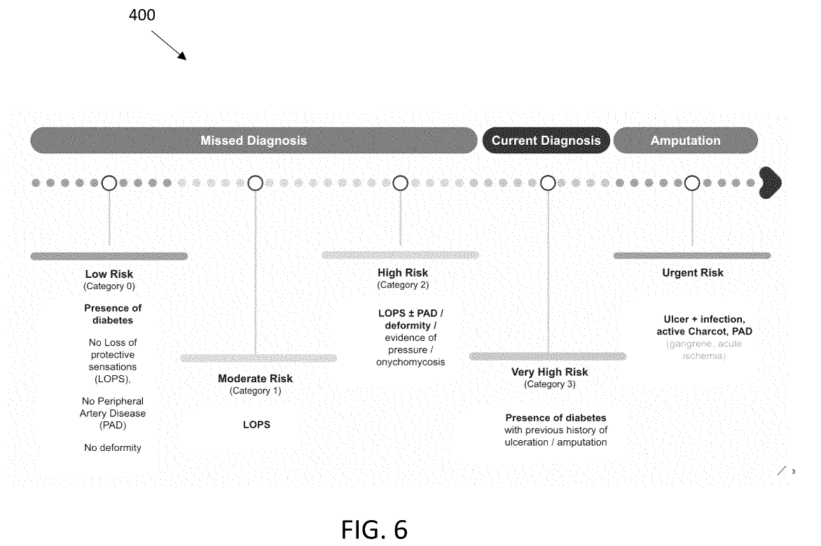

FIG. 6 shows a chart 400 of early vs. late-stage diabetic foot ulcer detection in a clinical validation wherein multiple trials were conducted to validate the thermal imaging system use. The trials drew a sample from the local population. Research findings show significant advancements in two areas. First, utilizing artificial intelligence to forecast the outcomes of a human thermographer's readings with a specificity 0.99 and sensitivity of 0.94. Second, a successful approach to differentiate between healthy and patients with diabetes. Using the thermal imaging system, abnormal foot readings were detected in 32% of the diabetic group, compared with only 1% observed in the healthy group. This figure aligns with the documented prevalence of foot ulcers among diabetic patients. The capability of the thermal imaging system to detect these abnormalities before they become visible to the naked eye improves the ulcer detection process.

The process used in implementing the thermal imaging system, mirrors the simplicity of taking a standard photograph and is highly user-friendly, making it suitable for both clinical and in-home care. Accessibility promotes widespread adoption across healthcare facilities, even in remote or underserved areas, ensuring that patients receive prompt and high-quality foot ulcer assessments.

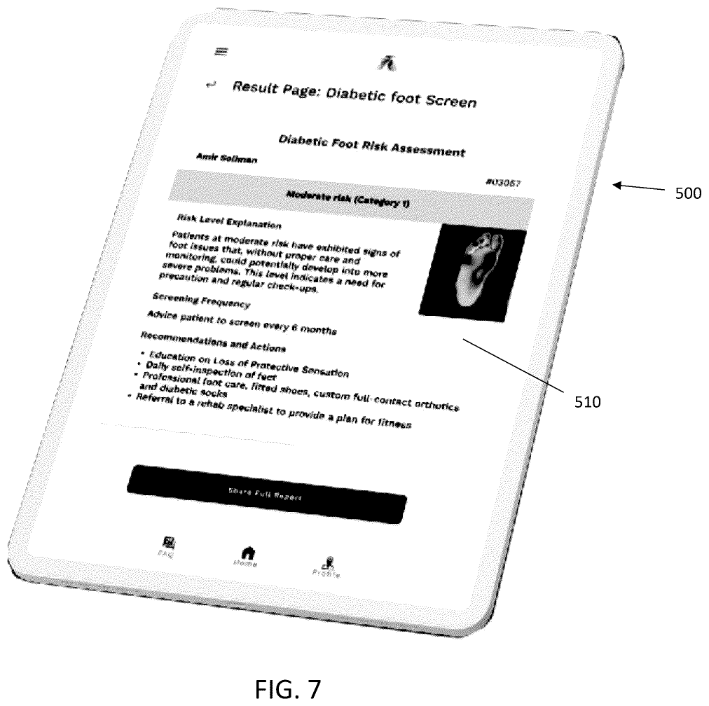

The mobile device shown in FIG. 7 is an example of a user GUI which may be a tablet 500 wherein the mobile device includes a graphical user interface 510 for displaying information and a computer medium for storing information and a processor for processing information for analysis of feet of a patient, the smart phone 300 in communication with a thermal imaging camera for capturing the thermal images of the feet of the patient. The outcome of the test may be displayed on any device having a graphical interface, with the user submits a thermal image directly from the thermal camera or via a smartphone/tablet wherein a pdf or other file can be displayed on the device or integrated into EMRs. The thermal imaging system uses advanced machine learning models in our data warehouse that analyze these thermal images wherein algorithms are specifically designed to detect variations in temperature and patterns that indicate potential health issues. By processing data with high accuracy, the thermal imaging system ensures its technology remains at the cutting edge of medical imaging and AI.

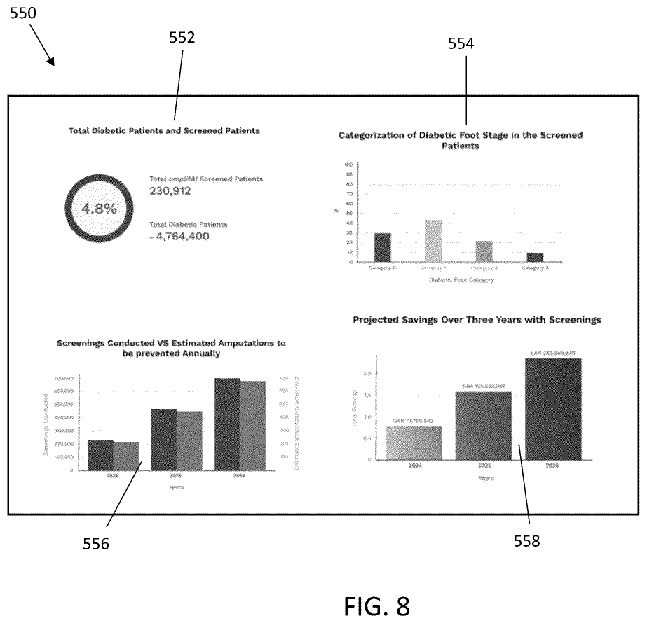

AI-driven analysis delivers rapid results, reducing the need for extensive medical expertise and thereby easing the workload of healthcare professionals. AI processed thermograms are used to carry asymmetry and analysis and detect abnormalities faster . . . etc. Following analysis, the software generates a detailed report, as illustrated on the dashboard of FIG. 8 and displayed on a device such as the tablet of FIG. 7, the report generated for each clinic, hospital or government to show the results of the aggregate data. The report provides crucial information such as the categorization of the diabetic foot condition, risk level, recommended screening frequency, and suggested actions. Designed for easy integration with healthcare systems, the report enables medical professionals to seamlessly incorporate the findings into the patient's care plan. This technology aims to guide interventions for foot care in patients with diabetes. The thermal imaging system is provided as an extension of the physical exam. Additional assessments are necessary to confirm the vascular or neuropathic disease and to grade the foot ulcers. The graphical user interface 310, 510 of the smartphone, tablet or other graphical device, shows an interactive dashboard used for aggregate data viewing. The information shown on the device may be a pdf or other file format. The information may also be integrated into health records or may be printed. The interactive dashboard provides a user-friendly interface designed to enhance your experience in accessing and interpreting the results. The interactive dashboard enables the user to efficiently analyze and explore the aggregated data generated by the thermal imaging system, contributing to informed decision-making and proactive healthcare management. The interfaces shown are samples of the interactive dashboard used in the thermal imaging system. The interactive dashboard includes section related to economic impact, social impact and results produced by the application.

Economic Impact-Leveraging thermography as a screening modality for DFUs has already demonstrated a noteworthy cost-saving potential. Despite the requirement for human thermographers in their methodology, their study projected substantial savings through the integration of thermography as a standard procedure. In light of these achievements, one can only imagine the transformative potential offered by the thermal imaging system wherein technology not only streamlines the thermographic process but also obviates the need for human intervention altogether. The prospective advantages and cost-saving implications of the thermal imaging system in the context of DFU screening are highly promising. With the prevalence of diabetic foot conditions on the rise, placing an escalating strain on healthcare systems, the efficiency and accessibility offered by the thermal imaging system hold the promise of effecting a significant and positive impact, both in terms of patient outcomes and healthcare economics.

A retrospective study estimated high annual costs of managing DFUs. A current study further contributes to this understanding, stating that on the order of one-third of the direct costs of diabetes are attributable to care for diabetic foot disease. Considering these findings and the assumption that up to one-third of individuals with diabetes may develop DFUs, it can be estimated that a significant portion of national healthcare burdens and may be attributed to DFUs.

FIG. 8 shows a visual representation of an economic impact dashboard 550 including a patient tally section 552, a categorized screen patient section 554, a screenings vs. amputations section 556 and a projected savings section 558. The economic impact section provides essential insights, encompassing statistics on the percentage of diabetic patients who underwent foot screening, risk severity categorization, and projected cost savings. The economic impact dashboard 550 shows a visual representation and serves as a speculative projection, using estimated data to illustrate the potential financial benefits of the diabetic foot screening technology.

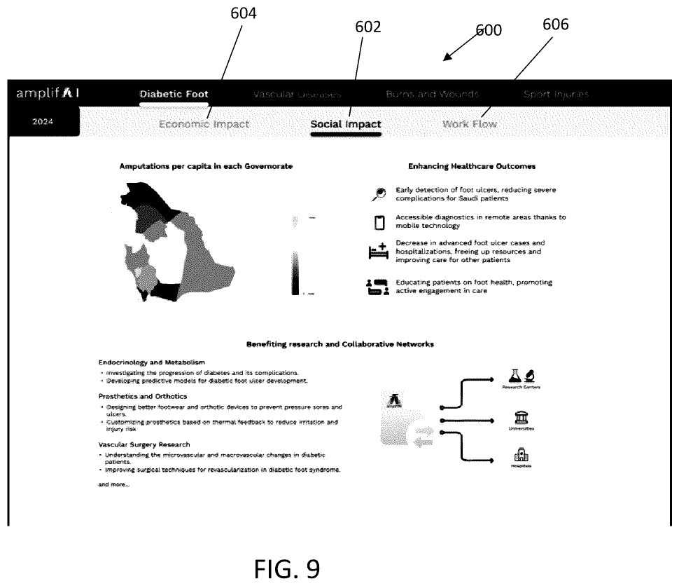

FIG. 9 shows a visual representation 600 of a social impact dashboard 604 wherein a social impact section presents a conceptual model of the potential benefits derived from implementation of the thermal imaging system. GPS coordinates are gathered to enable a tailored approach, customizing findings to align with specific health clusters across the entire country. The figures forecast the possible enhancements in healthcare outcomes such as the accessibility of diagnostics in remote areas and the educational impact on patient self-care.

The thermal imaging system includes the thermal imaging system which represents an advancement in diabetic foot care including potential to transform the early detection of DFUs offers significant benefits and including cost savings, improved accessibility, and streamlined healthcare workflows. The workflow is included on the interactive dashboard as shown in the workflow tab 606 of FIG. 9. The economic impact tab 602 and the social impact tab 604 are also shown with the social impact tab 604 being the active tab in the visual representation.

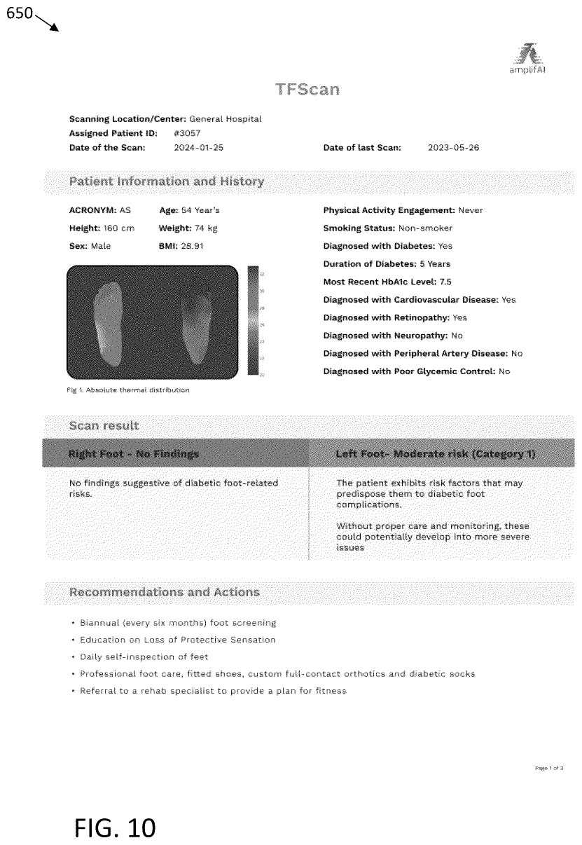

FIG. 10 shows data from a patient scan report including patient information and history, scan results, and recommendations and actions. FIG. 11 shows a non-thermal picture of a patient's feet.

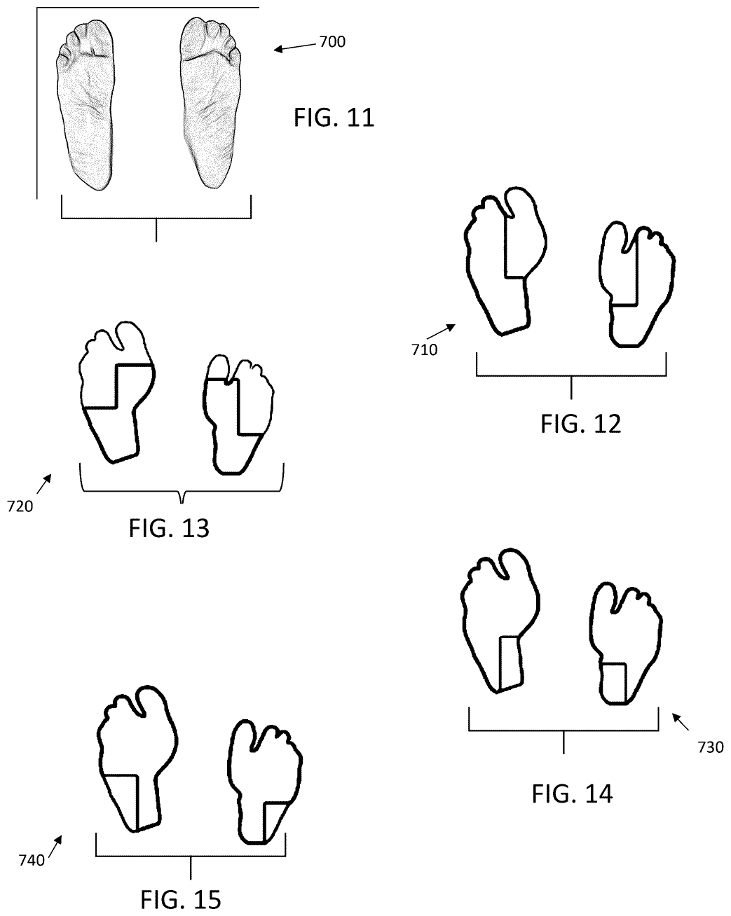

FIGS. 12-15 show temperature conditioning index (TCI) showing −4.4° MPA 710 in FIG. 12, −4.6° LPA 720 in FIG. 13, −1.9° MCA 730 in FIG. 14 and −1.9° LCA 740 in FIG. 15. TCI right average is 26.5, left average is 30.3 and the difference between right and left average is −3.8.

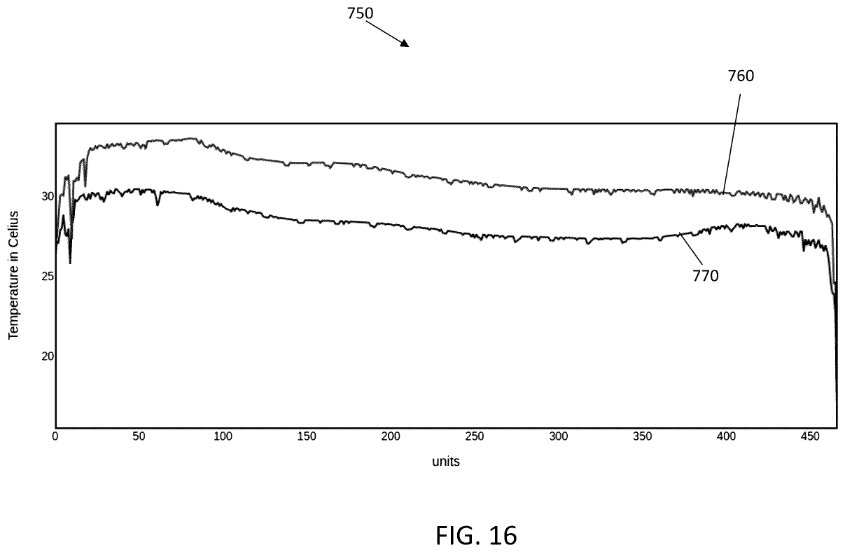

FIG. 16 is graph 800 of Paco's Curve 750 representation of a patient's right foot 760 and left foot 770. The Paco curve serves as a graphical representation within the realm of podiatric thermography, delineating the mean temperature profile along the vertical axis of a linear thermogram extending from the toe to the calcaneus. Primarily employed for assessing the asymmetry in thermal distribution along a single axis between bilateral feet, Paco's curve offers valuable insights into interlimb temperature differentials. The thermal imaging system facilitates the automated generation of these Paco curves from processed thermograms, enhancing efficiency and objectivity in data analysis.

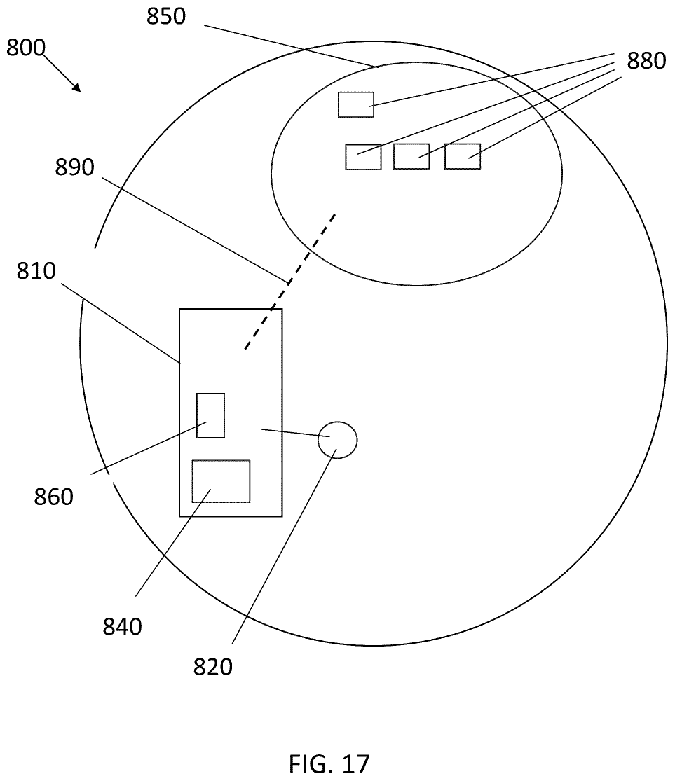

FIG. 17 is a diagram showing a preferred embodiment of the present thermal imaging system 800 for detecting diabetic foot ulcers. The system includes a mobile device 810 which includes a thermal camera 820 for obtaining thermal images. The system uses a neural network 850 in communication 890 with the mobile device 810, the mobile device 810 having an application for processing the obtained thermal images 840 and a set of instructions 860 for analyzing the thermal images 840. The neural network 850 include trained data 880 which allows the information sent to the neural network to further train the neural network 850 and improve accuracy of results returned to the mobile device 810 by the neural network 850.

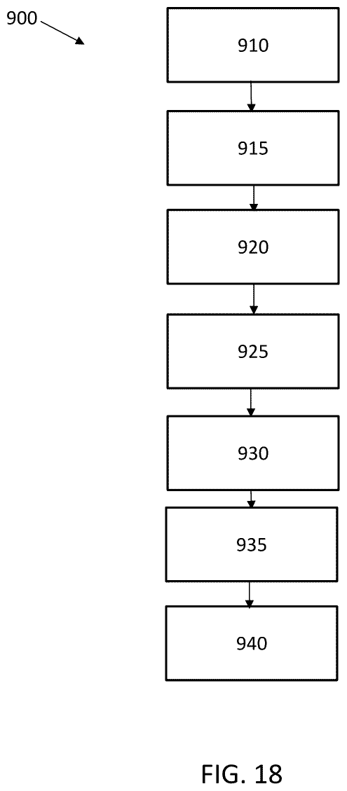

FIG. 18 is a flowchart 900 showing a method for using the thermal imaging system. The method includes initiating the mobile device application 905, obtaining patient information 910, obtaining scanned thermal images by scanning feet of the patient using the thermal camera 915, initializing analysis of the scanned thermal images using a temperature conditioning index 920, sending data to a neural network for neural network entry 925, analysis 930 and feedback 935, and the application providing patient results to the graphical user interface of the mobile device 940.

The system includes a reporting service which represents the lens of the healthcare professional Reports summarize the risk factors of the assessment protocol and offer a clear breakdown of our algorithmic assessment approach, complete with automated screening insights for future follow-ups with patients. The workflow of the reporting service is built upon established and validated screening protocols, including the widely adopted Canadian Inlow 60-sewcond foot screen.

In one embodiment of a system of analysis using a thermal foot scan for identifying diabetic foot complications, the system uses thermal cameras compatible with a mobile device for capturing detailed thermal images of patient feet. The system uses machine learning models for analyzing thermal images captured by the thermal camera, wherein algorithms on the mobile device detect variations in temperature and thermal patterns that indicate potential health issues, including processing large sets of data wherein the system ensures accuracy by implementing learned data from the machine learning models. The reporting service may deliver the data results to the patient, to a healthcare system database or to both.

In some embodiments the method or methods described above may be executed or carried out by a computing system including a tangible computer-readable storage medium, also described herein as a storage machine, that holds machine-readable instructions executable by a logic machine (i.e. a processor or programmable control device) to provide, implement, perform, and/or enact the above-described methods, processes and/or tasks. When such methods and processes are implemented, the state of the storage machine may be changed to hold different data. For example, the storage machine may include memory devices such as various hard disk drives, CD, or DVD devices. The logic machine may execute machine-readable instructions via one or more physical information and/or logic processing devices. For example, the logic machine may be configured to execute instructions to perform tasks for a computer program. The logic machine may include one or more processors to execute the machine-readable instructions. The computing system may include a display subsystem to display a graphical user interface (GUI) or any visual element of the methods or processes described above. For example, the display subsystem, storage machine, and logic machine may be integrated such that the above method may be executed while visual elements of the disclosed system and/or method are displayed on a display screen for user consumption. The computing system may include an input subsystem that receives user input. The input subsystem may be configured to connect to and receive input from devices such as a mouse, keyboard or gaming controller. For example, a user input may indicate a request that certain task is to be executed by the computing system, such as requesting the computing system to display any of the above-described information, or requesting that the user input updates or modifies existing stored information for processing. A communication subsystem may allow the methods described above to be executed or provided over a computer network. For example, the communication subsystem may be configured to enable the computing system to communicate with a plurality of personal computing devices. The communication subsystem may include wired and/or wireless communication devices to facilitate networked communication. The described methods or processes may be executed, provided, or implemented for a user or one or more computing devices via a computer-program product such as via an application programming interface (API).

Since many modifications, variations, and changes in detail can be made to the described embodiments of the invention, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of the invention should be determined by the appended claims and their legal equivalents.

In addition, the present invention has been described with reference to embodiments, it should be noted and understood that various modifications and variations can be crafted by those skilled in the art without departing from the scope and spirit of the invention. Accordingly, the foregoing disclosure should be interpreted as illustrative only and is not to be interpreted in a limiting sense. Further it is intended that any other embodiments of the present invention that result from any changes in application or method of use or operation, method of manufacture, shape, size, or materials which are not specified within the detailed written description or illustrations contained herein are considered within the scope of the present invention.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A thermal imaging system for detection of diabetic foot ulcers, the system comprising:
a mobile device including a graphical user interface;
a thermal camera in communication with the mobile device, the thermal camera for obtaining thermal images of patient feet and sending the thermal images to the mobile device;
an application on the mobile device;
wherein the application includes instruction for analyzing the thermal images using a temperature condition index during analysis; and
wherein the application provides data results on the graphical user interface.

2. The thermal imaging system according to claim 1 wherein the data results are provided on an interactive dashboard.

3. The thermal imaging system according to claim 2 wherein the interactive dashboard includes a current diagnosis.

4. The thermal imaging system according to claim 2 wherein the interactive dashboard incudes medical risk results including risk of amputation.

5. The thermal imaging system according to claim 1 wherein the thermal imaging device is securable to the mobile device.

6. The thermal imaging system according to claim 1 wherein the instruction for analyzing the thermal images include AI-driven analysis for delivering rapid results, reducing need for extensive medical expertise and easing workload of healthcare professionals.

7. The thermal imaging system according to claim 1 wherein following analysis, the application generates a detailed report including information on categorization of the diabetic foot condition, risk level, recommended screening frequency, and suggested actions.

8. The thermal imaging system according to claim 7 wherein the thermal imaging system is integratable with a healthcare system and wherein the report enables medical professionals to seamlessly incorporate results into a patient care plan.

9. The thermal imaging system according to claim 1 wherein the mobile application has an interactive dashboard including an economic impact dashboard, social impact dashboard, and an economic impact dashboard.

10. The thermal imaging system according to claim 1 wherein machine a learning model is accessible by the mobile device wherein machine learning algorithms analyze variations in temperature and image patterns which indicate potential health issues and wherein the machine learning models process large sets of data with high accuracy.

11. The thermal imaging system according to claim 1 wherein the system calculates risk factors and provides results of the risk factors on the graphical user interface.

12. The thermal imaging system according to claim 1 wherein a scan report is provided for a patient review or for further medical evaluation.

13. The thermal imaging system according to claim 1 wherein the system uses a Paco's Curve during the analysis.

14. A method of using the thermal imaging system according to claim 1, the method comprising:
initiating the mobile device application;
obtaining patient information;
obtaining scanned thermal images by scanning feet of the patient using the thermal camera;
initializing analysis of the scanned thermal images using a temperature conditioning index; and
the application providing patient results to the graphical user interface of the mobile device.

15. A method of using the thermal imaging system according to claim 1, the method comprising:
initiating the mobile device application;
obtaining patient information;
obtaining scanned thermal images by scanning feet of the patient using the thermal camera;
initializing analysis of the scanned thermal images using a temperature conditioning index;
the mobile device accessing a neural network wherein data obtained from the analysis of the scanned thermal images is sent to the neural network for comparison to learned data stored on the neural network and wherein the neural network provides feedback data to the mobile device for determining final analysis of patient results, the application providing the final patient results to the graphical user interface of the mobile device.

16. A thermal imaging system for detection of diabetic foot ulcers, the system comprising:
a computer medium including storage for patient information, patient history, scan results, recommendations and actions;
a computer processor for accessing the computer medium and for analyzing the patient history, scan results and to aid in preparing a course of action and recommendations;
a mobile device having a graphical user interface;
a thermal camera in communication with the mobile device, the thermal camera for obtaining thermal images and sending the thermal images to the mobile device;
wherein the system uses a temperature condition index during analysis.

17. A thermal imaging system for detection of diabetic foot ulcers, the system comprising:
a mobile device including a graphical user interface;
a thermal camera in communication with the mobile device, the thermal camera for obtaining thermal images of patient feet and sending the thermal images to the mobile device;
an application on the mobile device for processing thermology data obtained by the thermal camera;
a plurality of datasets accessible by the mobile device; and
a reporting service for delivering data results;
wherein the mobile device accesses at least one of the plurality of datasets; and
wherein the mobile device sends information to the at least one of the plurality of datasets.

18. The thermal imaging system according to claim 17 wherein the data results are provided on an interactive dashboard.

19. The thermal imaging system according to claim 17 wherein the interactive dashboard includes a current diagnosis.

20. A system of analysis using the thermal imaging system according to claim 1 to obtain a thermal foot scan for identifying diabetic foot complications, the system using thermal cameras, the thermal cameras compatible with a mobile device for capturing detailed thermal images of patient feet, the system using machine learning models for analyzing thermal images captured by the thermal camera, wherein algorithms on the mobile device detect variations in temperature and thermal patterns that indicate potential health issues, including processing large sets of data wherein the system ensures accuracy by implementing learned data from the machine learning models.
